(19) United States
(12) Patent Application Publication
(10) Pub. No.: US 2025/0064999 A1
(43) Pub. Date: Feb. 27, 2025

(54) Portable Decontamination Chamber

(71) Applicant: Government of the United States as Represented by the Secretary of the Army

(72) Inventors: Amee Polk, Havre de Grace, MD (US); Warren Gardner, Bel Air, MD (US); Michael Kauzlarich, Edgewood, MD (US); Todd Sickler, Phoenix, MD (US); Tobias Dilworth, Fulton, MD (US); Kathleen Craft, Savage, MD (US); Tessa VanVolkenburg, Ellicott City, MD (US); Ashley Lakey, Ashton, MD (US); Joseph May, Ocklawaha, FL (US)

(21) Appl. No.: 18/638,758

(22) Filed: Apr. 18, 2024

Related U.S. Application Data
(60) Provisional application No. 63/578,503, filed on Aug. 24, 2023.

(51) Int. Cl. A61L 2/04; A61L 2/26; C06B 33/12; F42B 3/11; F42B 3/113

(52) U.S. Cl. CPC A61L 2/04; A61L 2/26; C06B 33/12; F42B 3/11; F42B 3/113; A61L 2202/121; A61L 2202/122; A61L 2202/16

(57) ABSTRACT

A portable decontamination chamber including an outer container defining a first internal volume; an interior receptacle positioned within the first internal volume and defining a second internal volume that is configured to receive an item to be decontaminated; an insulating liner configured to be positioned within the first internal volume between an interior surface of the outer container and an exterior surface of the interior receptacle; a thermite compact configured to be positioned within the first internal volume and in contact with at least a portion of the exterior surface of the interior receptacle; and an initiator configured to ignite the thermite compact to induce a biological decontamination temperature within the second internal volume in order to thoroughly and rapidly decontaminate material tainted with unwanted biological matter in a readily deployable package without the generation of additional gaseous products that increase the pressure within the decontamination chamber.

### RELATED APPLICATIONS

This application is a non-provisional application that claims priority benefit of U.S. Provisional Application Ser. No. 63/578,503 filed Aug. 24, 2023; the contents of which are hereby incorporated by reference.

### GOVERNMENT INTEREST

The invention described herein may be manufactured, used and licensed by or for the U.S. Government.

### FIELD OF THE INVENTION

The present invention relates to a decontamination chamber, and more particularly to a decontamination chamber that is able to thoroughly and rapidly decontaminate material tainted with unwanted biological matter (including DNA fragments, prions, microbes, and spores) in a readily deployable package.

### BACKGROUND OF THE INVENTION

Sterilization, disinfection, sanitization, and decontamination apparatus and methods are used in a broad range of applications in order to decontaminate material tainted with unwanted biological matter. A variety of methods are used, including steam, chemicals, fumigants, radiation, among others. Drawbacks to these methods exist, and are addressed by the devices, systems and methods disclosed herein.

Traditional thermite is an incendiary mixture that, while not an explosive material, can burn very fast and very hot. However, traditional thermite reactions generate vaporous products during combustion and therefore in a closed vessel create a great deal of pressure above that due to gas expansion during heating. Additionally, these gas generating thermites have high burn rates due to the increased thermal conductivities of the gas phase constituents. As such, traditional thermites have not been feasible for use in portable decontamination devices.

In contrast, non-traditional thermite (NTT) pyrotechnic compositions can reach temperatures upwards of 1,500° C. for an extended length of time due to their slow burn rates, allowing the biological material to be inactivated/decontaminated by the heat. Recent research by Flax et al (2022) demonstrated dry heat decontamination on the representative organism Bacillus atrophaeus spores required <4.63 minutes per log decrease at 170° C.

Thus, there exists a need for a decontamination chamber that leverages the advantageous characteristics of thermite in order to thoroughly and rapidly decontaminate material tainted with unwanted biological matter (including DNA fragments, prions, microbes, and spores) using a dry heat approach in a readily deployable package.

### SUMMARY OF THE INVENTION

The present invention provides a portable decontamination chamber that includes an outer container defining a first internal volume; an interior receptacle positioned within the first internal volume and defining a second internal volume that is configured to receive an item to be decontaminated; an insulating liner configured to be positioned within the first internal volume between an interior surface of the outer container and an exterior surface of the interior receptacle; a thermite compact configured to be positioned within the first internal volume and in contact with at least a portion of the exterior surface of the interior receptacle; and an initiator configured to ignite the thermite compact to induce a biological decontamination temperature within the second internal volume in order to thoroughly and rapidly decontaminate material tainted with unwanted biological matter in a readily deployable package without the generation of additional gaseous products that increase the pressure within the decontamination chamber.

### BRIEF DESCRIPTION OF THE DRAWINGS

The present invention is further detailed with respect to the following figures that depict various aspects of the present invention.

FIG. 1 is a cross sectional view of a decontamination chamber according to embodiments of the present invention;

FIG. 2 is a top view of a decontamination chamber with the lid removed according to embodiments of the present invention;

FIG. 3 is a bottom view of the lid of a decontamination chamber removed from the body of the decontamination chamber according to embodiments of the present invention;

FIG. 4 is a top view of a lid of a decontamination chamber according to embodiments of the present invention; and

FIG. 5 is a graph showing thermocouple data collected from the interior of three decontamination chambers and the exterior of one decontamination chamber during testing.

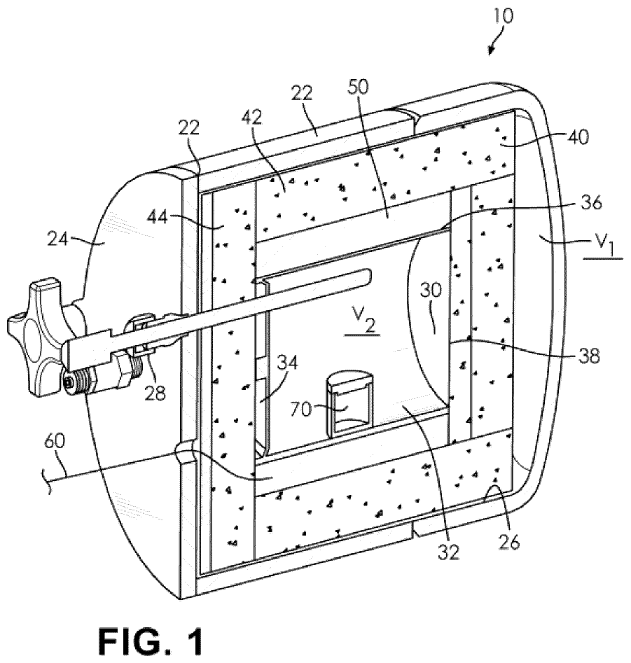

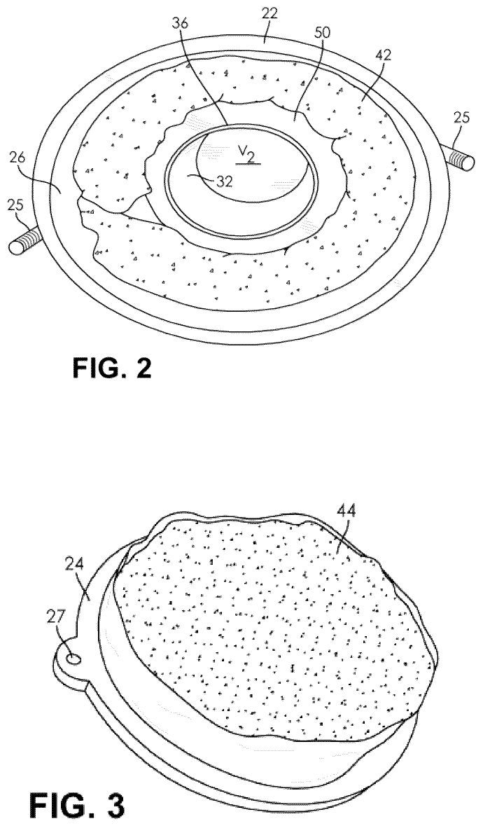

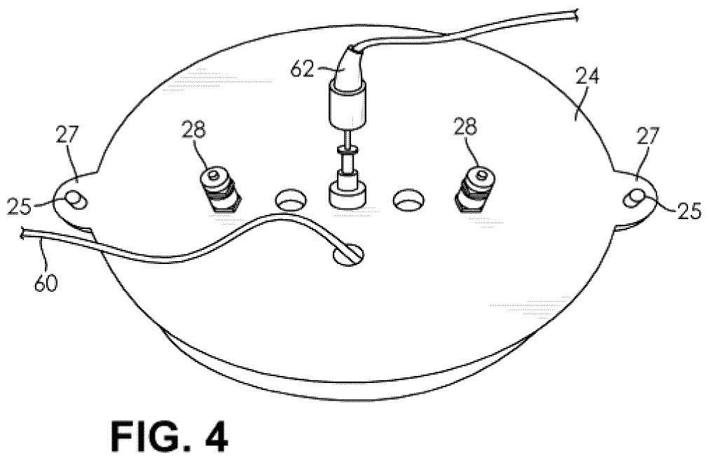

### DESCRIPTION OF THE INVENTION

The present invention has utility as a decontamination chamber that leverages the advantageous characteristics of thermite in order to thoroughly and rapidly decontaminate material tainted with unwanted biological matter (including viruses, bacteria, proteins, toxins, DNA fragments, prions, microbes, and spores) in a readily deployable package. The present invention utilizes non-traditional oxidizer-based thermites to inactivate biological samples using a dry heat approach both in terrestrial and space-based applications.

This device is designed to thoroughly and rapidly decontaminate material tainted with unwanted biological matter (including DNA fragments, prions, microbes, and spores) in a readily deployable package. Potential applications include space flight and astrobiological life detection, mobile decontamination and destruction of biological (virus, bacteria, prions, proteins, and toxins) samples associated with naturally occurring isolates, and biosecurity (bioterrorism and biological defense). The inventive device requires no outside power to operate (excepting the igniter to initiate the thermite reaction) and can therefore be deployed in a variety of austere scenarios. The inventive decontamination chamber is thus easy to use, portable, and safe for the operator.

The recent development of Non-Traditional Thermites (NTT) promises to expand the use of these safe, high energy pyrotechnic compositions into new areas of use. NTT pyrotechnic compositions possess the high exothermic heat of reaction of traditional thermite compositions except the generation of vaporous products due to the chemical reaction. This allows the NTT composition to provide a significant amount of heat to the target biological material without the generation of additional gaseous products that increase the pressure within the decontamination vessel. NTT compositions form thermal insulating combustion products which allow the maximum amount of thermal transfer to the target. Initial tests of NTT compositions against biological material show complete inactivation of the biological material with no damage to the containment device. The absence of additional pressure generation allows for the inventive decontamination chamber 10 to remain a completely closed, isolated system with no concerns for rupture due to overpressure. This approach successfully destroys biological samples while maintaining the structural integrity of the device and preventing excessive heat transfer to the exterior of the decontamination chamber.

The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment may be incorporated into other embodiments, and features illustrated with respect to a particular embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Components and features described herein may be combined in any desired manner to achieve the desired performance goals.

According to embodiments and as shown in the figures, a portable decontamination chamber 10 includes an outer container 20, an interior receptacle 30, an insulating liner 40, a thermite compact 50, and an initiator 60. The outer container 20 has a body 22 and a removable lid 24 and defines a first internal volume V₁. The interior receptacle 30 has a body 32 and a removable lid 34 and defines a second internal volume V₂ that is configured to receive an item to be decontaminated 70. The interior receptacle 30 is configured to be positioned within the first internal volume V₁ of the outer container 20, and according to embodiments is removable from the outer container 20. The insulating liner 40 is configured to be positioned within the first internal volume V₁ between an interior surface 26 of the outer container 20 and an exterior surface 36 of the interior receptacle 30. The thermite compact 50 is configured to be positioned within the first internal volume V₁ and in contact with at least a portion of the exterior surface 36 of the interior receptacle 30. The initiator 60 is configured to ignite the thermite compact 50 to induce a biological decontamination temperature within the second internal volume V₂.

As noted above, the outer container 20 has a body 22 and a removable lid 24 and defines a first internal volume V₁. According to some embodiments, the outer container 20 is formed of a thermally resistant material, such as refractory metals, including Niobium, Molybdenum, Tantalum, Tungsten, and Rhenium; glass; ceramic; silicon carbide; aluminum nitride; steel; stainless steel, titanium; high temperature carbon fiber composites; or a combination thereof. According to some embodiments, the outer container 20 is formed of a material that is not thermally resistant, given that temperatures measured outside of the outer container 20 generally remain less than 200° C. It was found that our formulation burns at about 1400° C., but that the exterior temperature of the outer container never exceeded 60° C. According to embodiments, the outer container 20 has an outer diameter of 6 to 12 inches and a height of 12 to 24 inches. According to embodiments, the wall that makes up the outer container 20 has a thickness of 0.1 to 0.5 inches. According to embodiments, the outer container includes at least two fasteners 25 extending from the body 22 of the container 20 that are configured to engage with through holes 27 in the lid 24 of the container in order to secure the lid 24 to the body 22 to seal the outer container 20; however, it will be understood that the body 22 and lid 24 of the container 20 may be joined using any other suitable means, including, but not limited to, cooperating threads on the lid 24 and body 22, toggle latches, swing bolts, etc., According to embodiments, the body 22 and the lid 24 include threads that engage with one another in order to secure the lid 24 to the body 22 to seal the outer container 20.

As noted above, the interior receptacle 30 has a body 32 and a removable lid 34 and defines a second internal volume V₂ that is configured to receive an item to be decontaminated. According to embodiments, the interior receptacle 30 is formed of a material that may be melted by the thermite reaction. According to some embodiments, the interior receptacle 30 is formed of a material that has inherent biocidal properties, including steel, aluminum, copper, brass, zinc, or a combination thereof. According to embodiments, the interior receptacle 30 has an outer diameter that is ¼ to ¾ the size of the outer diameter of the outer container 20. According to embodiments, the wall that makes up the interior receptacle 30 is very thin, e.g. about 12 gauge or 7/64″ or less.

As noted above, the insulating liner 40 is configured to be positioned within the first internal volume V₁ between an interior surface 26 of the outer container 20 and an exterior surface 36 of the interior receptacle 30. The insulating liner 40 provides thermal insulation to both protect the outer container 20 and contain the heat from the ignited thermite compact 50. According to embodiments, the insulating liner 40 is formed of alumina, silica, or a combination thereof. According to embodiments, the insulating liner 40 is a batting material. According to embodiments, insulating liner 40 includes a body portion 42 that surrounds the body 32 of the interior receptacle 30 and a removable lid portion 44 that is configured to be positioned between the lid 34 of the interior receptacle 30 and the lid 24 of the outer container 20. According to embodiments, the insulating liner 40 is removeable and replaceable.

As noted above, the thermite compact 50 is configured to be positioned within the first internal volume V₁ and in contact with at least a portion of the exterior surface 36 of the interior receptacle 30. According to embodiments, the thermite compact 50 is provided surrounding the exterior wall 36 of the interior receptacle 30 and/or is positioned along a bottom 38 of the interior receptacle 30. The thermite of the thermite compact 50 is a non-traditional thermite (NTT) pyrotechnic composition and can reach temperatures upwards of 1,500° C. for an extended length of time due to its slow reaction rate, allowing the biological material to be inactivated/decontaminated by the heat. When activated, the thermite undergoes a rapid exothermic reaction, heating the contents within the second volume V₂ of the interior receptacle 30 and decontaminating any biological agents that may be present. Biological materials inside the second volume V₂ of the interior receptacle 30 that are sensitive to high heat (temperature of >500° C.) will be acted upon by the thermite reaction.

The thermite used as the thermite compact 50 of the present invention has several advantages over alternative formulations. It is comparatively low outgassing (little overpressure produced within the vessel, limiting potential for material to escape), nonexpanding, long-reacting, environmentally safe, and mechanically stable. According to embodiments, the thermite material is non-expanding and produces low or no outgassing such that a pressure within the second internal volume V₂ does not increase upon ignition of the thermite compact 50. The thrust here is that no vaporous products are generated. A slight pressure increase may be expected because the thermite heats the ambient air, but this volume expansion is able to be contained by the vessel. We calculated a maximum of 5× expansion due to heat via the ideal gas law, which would be worst case. Upon cooling the chamber would return to about the original internal pressure. Technically, a volume within the chamber referred to herein as internal volume V₂ increases as the air in the interior is heated. When it cools, it returns to nominal pressure. Importantly, the thermite material does not generate any products that increase the total quantity of gas contained within the vessel. According to embodiments, the thermite compact 50 is formed of a thermite material comprising by atomic percent a mixture of 40 to 50 percent titanium dioxide and 50 to 60 percent magnesium, aluminum, and zirconium, or combination thereof. According to embodiments, the thermite compact 50 is formed of a thermite material comprising by atomic weight 20 to 60 percent metal and between 40 to 80 percent metal oxide, e.g., TiO₂. The percentages presented herein do not include a binder unless specifically noted. According to embodiments, the thermite material additionally comprises by atomic weight up to 1 to 30 percent of a metallic or rubberized binder including Hytemp rubber, stearic acid, nitrocellulose, PVA, and indium, which adjusts the consistency of the thermite material. According to embodiments, the thermite material that forms the thermite compact 50 additionally includes iodine or iodine pentoxide. The ratios of the components can be varied in such a manner as to adjust the overall rate and/or temperature of the chemical reaction which is represented by the burning rate for the thermite compact 50. This non-traditional thermite composition is a robust formulation; a variation of the component ratio of +2% does not affect its function. The specific tested formulation for the trials described in the following examples includes 27% aluminum, 27% zirconium, and 46% titanium dioxide by atomic ratio, with up to 10% total formulation mass of binder.

According to embodiments, the non-traditional thermite is pressed into a compact 50 at a loading pressure between 2500 and 5000 pounds per square inch. According to embodiments, the thermite is pressed into various shapes for use with the decontamination device. For example, the thermite compact 50 may be formed as either a ring that sits around the exterior surface 36 of the internal receptacle 30 or the thermite compact 50 may be pressed directly into the inside of the internal receptacle 30.

As noted above, the initiator 60 is configured to ignite the thermite compact 50 to induce a biological decontamination temperature within the second internal volume V₂. According to embodiments, the initiator 60 is an electrically based resistively heated filament ignitor, which according to embodiments is a tungsten filament. According to embodiments, the initiator 60 is an opto-pyrotechnic laser diode initiator.

The inventive portable decontamination chamber 10 may be operated in either a vertical or horizontal position depending on the materials to be loaded into the device. The lid 24 with the insulating liner 40 is used to seal the container 20. According to some embodiments, an overpressure release valve 28 is present in the outer container 20 for additional safety and is configured to fluidly communicate the first internal volume V₁ with an exterior environment when a pressure within the first internal volume V₁ exceeds a threshold pressure. The portable decontamination chamber 10 is configured to also contain the initiator 60 and thermocouples 62 (internal and external) to monitor the heat post ignition. According to embodiments, additional holes are provided in the outer container 20 for the incorporation of high efficiency particulate air (HEPA) filter and/or other sensors. According to embodiments, the portable decontamination chamber 10 has a hazardous division rating no higher than 1.3 for transportation.

### Example 1

Dry heat resistance studies are conducted using a spore preparation of Bacillus atrophaeus NRRL B4418 (Steris, NA026). Sterile 304 stainless steel coupons (22 mm×22 mm×1 mm) are inoculated with 0.1 ml of 3.63×10⁸ cfu/ml B. atrophaeus spores. The coupons are dried over four hours in a Type II B2 Biological Safety Cabinet. The inoculated coupons are then loaded into three enclosed coupon holders that are then placed into the second volume V₂ within the interior receptacle 30 of the decontamination chamber 10, each containing 10 coupons per chamber. Three additional inoculated coupons are used to determine percent recovery off a stainless-steel coupon compared to the starting stock spore solution.

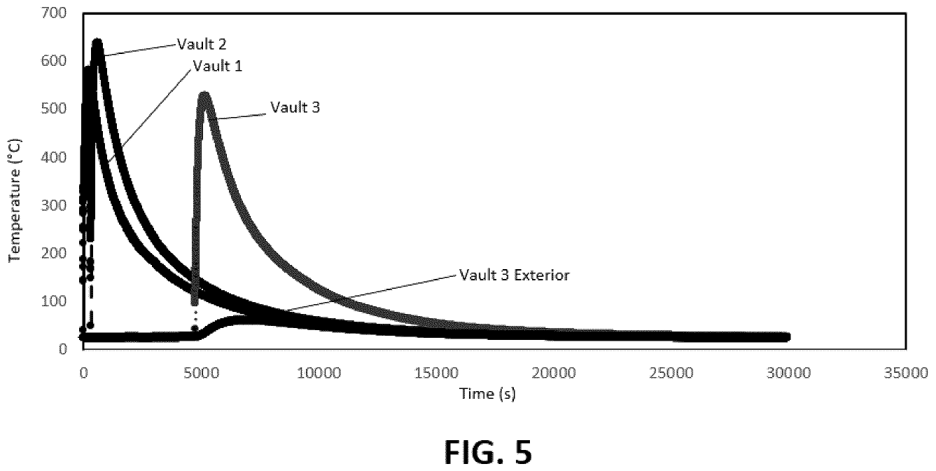

FIG. 5 shows the temperature profile collected from thermocouple data for each of the three decontamination chambers, indicating all test replicates reached far greater than sufficient temperature to destroy the biological material. Table 1 gives the analysis of the biological samples post exposure to the NTT. Plates exceeding 300 colony forming units are labelled too numerous to count (TNTC).

TABLE 1
Biological data of coupons spotted with 3.63 × 10⁷ Bacillus atrophaeus spores
grown at 35° C. on Tryptic Soy Agar plates after serial dilution
in sterile Cytiva HyClone HyPure water.

| Sample/ | Dilution Series (# of Colonies, triplicate plates) | | | | | | | Concen- | Coupon |
|---|---|---|---|---|---|---|---|---|---|
| Coupon # | 0 | −1 | −2 | −3 | −4 | −5 | −6 | tration | Holder # |
| (−) control | 0/0/0 | | | | | | | | |
| (+) 1 control | | | | 94/89/76 | 18/25/21 | 1/0/3 | 0/0/1 | 8.63 × 10⁵ | |
| dried coupon | | | | | | | | CFU/mL | |
| (+) 2 control | | | | 241/244/267 | 22/27/20 | 4/5/2002 | 0/0/1 | 2.51 × 10⁶ | |
| dried coupon | | | | | | | | CFU/mL | |
| (+) 3 control | | | | 157/154/184 | 21/31/17 | 7/0/3 | 0/0/1 | 1.65 × 10⁶ | |
| dried coupon | | | | | | | | CFU/mL | |
| (+) 4 control | | | | | | TNTC | 38/38/33 | 3.63 × 10⁸ | |
| Stock | | | | | | | | CFU/mL | |
| 1 | 0/0/0 | 0/0/0 | 0/0/0 | 0/0/0 | 0/0/0 | | | | 1 |
| 2 | 0/0/0 | 0/0/0 | 0/0/0 | 0/0/0 | 0/0/0 | | | | 1 |
| 3 | 0/0/0 | 0/0/0 | 0/0/0 | 0/0/0 | 0/0/0 | | | | 1 |
| 4 | 0/0/0 | 0/0/0 | 0/0/0 | 0/0/0 | 0/0/0 | | | | 1 |
| 5 | 0/0/0 | 0/0/0 | 0/0/0 | 0/0/0 | 0/0/0 | | | | 1 |
| 6 | 0/0/0 | 0/0/0 | 0/0/0 | 0/0/0 | 0/0/0 | | | | 1 |
| 7 | 0/0/0 | 0/0/0 | 0/0/0 | 0/0/0 | 0/0/0 | | | | 1 |
| 8 | 0/0/0 | 0/0/0 | 0/0/0 | 0/0/0 | 0/0/0 | | | | 1 |
| 9 | 0/0/0 | 0/0/0 | 0/0/0 | 0/0/0 | 0/0/0 | | | | 1 |
| 10 | 0/0/0 | 0/0/0 | 0/0/0 | 0/0/0 | 0/0/0 | | | | 1 |
| 11 | 0/0/0 | 0/0/0 | 0/0/0 | 0/0/0 | 0/0/0 | | | | 2 |
| 12 | 0/0/0 | 0/0/0 | 0/0/0 | 0/0/0 | 0/0/0 | | | | 2 |
| 13 | 0/0/0 | 0/0/0 | 0/0/0 | 0/0/0 | 0/0/0 | | | | 2 |
| 14 | 0/0/0 | 0/0/0 | 0/0/0 | 0/0/0 | 0/0/0 | | | | 2 |
| 15 | 0/0/0 | 0/0/0 | 0/0/0 | 0/0/0 | 0/0/0 | | | | 2 |
| 16 | 0/0/0 | 0/0/0 | 0/0/0 | 0/0/0 | 0/0/0 | | | | 2 |
| 17 | 0/0/0 | 0/0/0 | 0/0/0 | 0/0/0 | 0/0/0 | | | | 2 |
| 18 | 0/0/0 | 0/0/0 | 0/0/0 | 0/0/0 | 0/0/0 | | | | 2 |
| 19 | 0/0/0 | 0/0/0 | 0/0/0 | 0/0/0 | 0/0/0 | | | | 2 |
| 20 | 0/0/0 | 0/0/0 | 0/0/0 | 0/0/0 | 0/0/0 | | | | 2 |
| 21 | 0/0/0 | 0/0/0 | 0/0/0 | 0/0/0 | 0/0/0 | | | | 3 |
| 22 | 0/0/0 | 0/0/0 | 0/0/0 | 0/0/0 | 0/0/0 | | | | 3 |
| 23 | 0/0/0 | 0/0/0 | 0/0/0 | 0/0/0 | 0/0/0 | | | | 3 |
| 24 | 0/0/0 | 0/0/0 | 0/0/0 | 0/0/0 | 0/0/0 | | | | 3 |
| 25 | 0/0/0 | 0/0/0 | 0/0/0 | 0/0/0 | 0/0/0 | | | | 3 |
| 26 | 0/0/0 | 0/0/0 | 0/0/0 | 0/0/0 | 0/0/0 | | | | 3 |
| 27 | 0/0/0 | 0/0/0 | 0/0/0 | 0/0/0 | 0/0/0 | | | | 3 |
| 28 | 0/0/0 | 0/0/0 | 0/0/0 | 0/0/0 | 0/0/0 | | | | 3 |
| 29 | 0/0/0 | 0/0/0 | 0/0/0 | 0/0/0 | 0/0/0 | | | | 3 |
| 30 | 0/0/0 | 0/0/0 | 0/0/0 | 0/0/0 | 0/0/0 | | | | 3 |

1-30 correspond to the test coupon samples. Each coupon is stainless steel and has B. atrophaeus spores spotted on it. Dilution series describes the log dilution from the starting solution. # of colonies represents viable spores. Triplicate describes how many samples at each dilution were sampled. In counting bacteria on standard agar plates, the general 30-300 colonies/plate rule is used. So <30 colonies and >300 colonies have increased error for plating error and undercounting. The concentration is what the starting solution is for that series when back calculated. Coupon holder describes which vessel was being tested. We repeated the test three times with 10 coupons/coupon holder for a total of 30 samples. The 0 dilution series is the starting solution after the samples were vortexed.

Patent documents and publications mentioned in the specification are indicative of the levels of those skilled in the art to which the invention pertains. These documents and publications are incorporated herein by reference to the same extent as if each individual document or publication was specifically and individually incorporated herein by reference.

The foregoing description is illustrative of particular embodiments of the invention but is not meant to be a limitation upon the practice thereof. The following claims, including all equivalents thereof, are intended to define the scope of the invention.

1. A portable decontamination chamber comprising:
an outer container having a body and a removable lid, the outer container defining a first internal volume;
an interior receptacle having a body and a removable lid, the interior receptacle defining a second internal volume that is configured to receive an item to be decontaminated, the interior receptacle configured to be positioned within the first internal volume;
an insulating liner configured to be positioned within the first internal volume between an interior surface of the outer container and an exterior surface of the interior receptacle;
a thermite compact configured to be positioned within the first internal volume and in contact with at least a portion of the exterior surface of the interior receptacle; and
an initiator configured to ignite the thermite compact to induce a biological decontamination temperature within the second internal volume.

2. The portable decontamination chamber of claim 1 wherein the outer container is formed of a thermally resistant material.

3. The portable decontamination chamber of claim 1 wherein the interior receptacle is formed of a material including steel, aluminum, copper, brass, zinc, or a combination thereof.

4. The portable decontamination chamber of claim 1 wherein the insulating liner is formed of alumina, silica, or a combination thereof.

5. The portable decontamination chamber of claim 1 wherein the insulating liner includes a body portion that surrounds the body of the interior receptacle and a removable lid portion that is configured to be positioned between the lid of the interior receptacle and the lid of the outer container.

6. The portable decontamination chamber of claim 1 wherein the thermite compact is provided surrounding the exterior wall of the interior receptacle.

7. The portable decontamination chamber of claim 1 wherein the thermite compact is positioned along a bottom of the interior receptacle.

8. The portable decontamination chamber of claim 1 wherein the thermite material is non-expanding and produces low or no outgassing such that a pressure within the second internal volume does not increase upon ignition of the thermite compact.

9. The portable decontamination chamber of claim 1 wherein the thermite compact is formed of a thermite material comprising by atomic percent a mixture of 40 to 50 percent titanium dioxide and 50 to 60 percent magnesium and aluminum or aluminum and zirconium.

10. The portable decontamination chamber of claim 1 wherein the thermite compact is formed of a thermite material comprising by atomic weight 20 to 60 percent metal and between 40 to 80 percent metal oxide.

11. The portable decontamination chamber of claim 1 wherein the thermite material additionally comprises by atomic weight up to 1 to 30 percent of a metallic or rubberized binder.

12. The portable decontamination chamber of claim 1 wherein the thermite material additionally comprises iodine or iodine pentoxide.

13. The portable decontamination chamber of claim 1 wherein the thermite material is consolidated to form the thermite compact at a loading pressure between 2500 and 5000 pounds per square inch.

14. The portable decontamination chamber of claim 1 wherein the initiator is an electrically based resistively heated filament ignitor.

15. The portable decontamination chamber of claim 14 wherein the resistively heated filament is a tungsten filament.

16. The portable decontamination chamber of claim 1 wherein the initiator is an opto-pyrotechnic laser diode initiator.

17. The portable decontamination chamber of claim 1 wherein the biological decontamination temperature is greater than 500° C.

18. The portable decontamination chamber of claim 1 further comprising an over pressure release valve in the outer container configured to fluidly communicate the first internal volume with an exterior environment when a pressure within the first internal volume exceeds a threshold pressure.

19. The portable decontamination chamber of claim 1 wherein the portable decontamination chamber has a hazardous division rating no higher than 1.3 for transportation.
